(19) United States
(12) Patent Application Publication
(10) Pub. No.: US 2022/0137027 A1
(43) Pub. Date: May 5, 2022

(54) METHOD FOR EVALUATING MOLECULAR CHANGES RELATED TO A MOLECULE EFFECT IN A BIOLOGICAL SAMPLE

(71) Applicant: IMABIOTECH, LOOS (FR)

(72) Inventors: JONATHAN STAUBER, LAMBERSART (FR); RIMA AIT-BELKACEM, ANTONY (FR); DAVID BONNEL, LAVENTIE (FR); FABIEN PAMELARD, ARMANTIERES (FR)

(21) Appl. No.: 17/424,509

(22) PCT Filed: Jan. 21, 2020
(86) PCT No.: PCT/EP2020/051388

§ 371 Date: Jul. 21, 2021

(30) Foreign Application Priority Data
Jan. 22, 2019 (EP) 19305081.2

(51) Int. Cl. G01N 33/50; G06T 7/00

(52) U.S. Cl. CPC G01N 33/5008; G06T 7/0012; G06T 2207/30096; G01N 2560/00; G06T 2207/10004; G01N 2570/00

(57) ABSTRACT

The present invention relates to a method for ex-vivo or in-vitro evaluation of an effect of a molecule of interest on at least one molecular marker in a dosed biological sample. The method of the invention is based on the segmentation of a biological sample based on the concentration of a molecule of interest and the comparison of molecular changes associated to the presence of the molecule of interest within different parts/segments of a same biological sample or between different biological samples, which may comprise different amounts of said molecule of interest and/or different molecules of interest.

### FIELD OF THE INVENTION

The present invention relates to a method to manually or automatically assess molecular changes over the concentration or intensity of at least one molecule of interest within a biological sample. More particularly, the present invention relates to a method wherein imaging technology is used for detecting changes relative to molecular biomarker(s) within a biological sample in connection with the presence of a molecule of interest (e.g., active compound). The present invention allows to define regions regarding activity compound location in order to calculate a score, a ratio or any values of molecular changes over the concentration of a molecule of interest.

The method of the invention finds its application in all domains involving the study of the behavior of a molecule of interest in a biological sample. The method of the invention can be advantageously used in proteomics, peptidomics, lipidomics, metabolomics, glycomics or pharmaceutics research in order to screen candidate molecules and evaluate their therapeutics or diagnostics potentials.

### BACKGROUND OF THE INVENTION

Development of active compounds in pharmaceutical, nutraceutical, agrochemical or cosmetic industries needs the identification of the mechanism of action (MoA), efficacy and toxicity in preclinical early stages in in vitro cellular models or later in in vivo animal models. Later these assays are developed and translated into clinical stages to validate drug efficacy and toxicity into human. Mass Spectrometry (MS), Immuno-HistoChemistry (IHC), Enzyme linked Immuno-sorbent Assay (ELISA), In Situ Hybridization (ISH), Polymerase Chain reaction (PCR) are the gold standard methods for molecular changes evaluation and quantification at different stages. All these methods have been developed to quantify molecules in samples, and to identify their role in drug efficacy or toxicity.

All these methods are commonly used to investigate the tissue response by evaluating and quantifying biomarkers of diagnosis, prognosis, efficacy or response to a treatment. When working with control and dosed (with active compound) samples, mass spectrometry is one of the most advanced methods since it does not need to tag the molecules of interest to be quantified. Mass spectrometry delivers molecular information about the biological impact of the active compound as an increase or a decrease of lipids, metabolites, peptides or proteins concentration in living systems. Other techniques (e.g., IF, IHC, ELISA, ISH, spectrophotometry or UV fluorescence) allow evaluating and quantifying peptides, proteins, RNA (Ribonucleic acids) and DNA (desoxyribonucleic acids) that are targeted using a tagged antibody or nucleic sequence that will bind to the targets.

In the pharmaceutical industry, pharmacodynamics is the domain where scientists investigate the impact of drugs at a certain time after drug dosing with the evaluation of targeted molecules (biomarkers) changes or untargeted molecules changes (to investigate drug response at a larger scale and to identify potential unknown compounds).

At the tissue level, all used methods described until now compare the tissue as a whole and this means that lots of informations are missing. These actual methods use relative or absolute quantification of the molecular changes in the whole tissue or in a tissue section and don't consider where the related active compound concentration is located in tissue, cells or other specific regions of interest. The methods don't consider the impact of the localization of the drug to its neighborhood.

At the cellular level, current cell viability-based measurements often lead to biased response estimates due to the analysis to all the cells without a link with the relative or absolute concentration of drug or active compounds that penetrate or enter the cells.

There is thus a need for a method that allow to correlate active compound concentration and biomarkers of efficacy with more accuracy and precision, in order to investigate the cellular response to an active compound.

### SUMMARY OF THE INVENTION

In this context, the inventors now propose a method that combines the identification and localization of a compound of interest (e.g., a drug candidate) into a target tissue to an image analysis of the molecular changes in the region(s) where the compound of interest is localized. Looking at the localization of the compound of interest and selecting regions where it is particularly localized and/or where it presents various concentrations, allows investigating at a very fine scale the response to said compound and the associated biomarkers changes. According to the invention, a comparison between different samples, or different regions of a same sample, leads to a knowledge of the molecular environment that may be associated to the presence and/or concentration of the compound of interest or not. The method of the invention also allows investigating the molecular impact of a compound of interest and scoring it by comparing different calculated ratios between the compound of interest and the associated biomarker(s) (e.g., marker(s) of efficacy or toxicity), leading to an understanding of the impact (e.g., efficacy or toxicity) of the compound of interest at the neighborhood level.

It is thus an object of the present invention to provide a method for ex-vivo or in-vitro evaluation of an effect of at least one molecule of interest on at least one molecular marker in a dosed biological sample comprising:
selecting a dosed biological sample, which has been previously exposed to the at least one molecule of interest;
detecting the presence of the at least one molecule of interest in the dosed biological sample with a molecular imaging method and obtaining a molecular map of the dosed biological sample for the at least one molecule of interest;
spatially segmenting the molecular map of the dosed biological sample as a function of spectral information to obtain a segmentation map of the at least one molecule of interest in the dosed biological sample;
selecting a first region of interest (ROI) from the segmentation map, said first ROI having a first intensity for the at least one molecule of interest;
measuring the intensity or quantity of at least one molecular marker in said first ROI;
comparing the intensity or quantity of at least one molecular marker in the first ROI with the intensity or quantity of at least one molecular marker in a second ROI, said second ROI being selected from the dosed biological sample or from another biological sample.

The method may be implemented with any kind of biological samples that may be analyzed with a molecular imaging method, including tissue samples, organoids, and biological fluid samples, such as urine sample, plasma sample, cerebrospinal fluid, and a cell suspension.

In a particular embodiment, the dosed biological sample is a tissue section which has been obtained by previously sampling an animal which has been previously administered by the molecule of interest. Alternatively, or in addition, the dosed biological sample has been contacted in vitro with the molecule of interest.

In a particular embodiment, the molecule of interest is a candidate molecule and the dosed biological sample has been previously obtained by sampling in an animal that has been previously administered with the candidate molecule.

In a particular embodiment, the second ROI is selected from the segmentation map of the dosed biological sample, said second ROI being physically different from the first ROI and having a second intensity for the molecule of interest. In another embodiment, the second ROI is selected from a second biological sample, which has been previously exposed to the molecule of interest at a dose concentration different from the dose concentration for the dosed biological sample, said second biological sample being from same biological origin as the dosed biological sample. Alternatively, the second ROI is selected from a second biological sample which has not been previously exposed to the molecule of interest, said second biological sample being from same biological origin as the dosed biological sample. In another embodiment, the second ROI is selected from a second biological sample which has been previously exposed to a second molecule of interest, said second biological sample being from same biological origin as the dosed biological sample.

In a particular embodiment, the comparison between the first and second ROI allows to identify at least one biological marker specific to the molecule of interest biological effect and response.

In a particular embodiment, the first and second ROIs have been contacted with two molecules of interest corresponding to two distinct candidate molecules, and the comparison between the first ROI and the second ROI allows to discriminate between said candidate molecules. In such embodiment, the biological markers compared in the first and second ROIs may be same or different.

In another embodiment, the first and second ROIs have been contacted with several (i.e., more than one) molecules of interest, that may be identical or distinct between the ROIs. In a particular embodiment, both ROIs have been contacted with the same two or more molecules of interest.

In a particular embodiment, the biological markers compared in the first and second ROIs are identical, even if the molecule of interest of the second ROI is different from the molecule of interest of the first ROI. The method of the invention may thus be used for evaluating and comparing the effect of at least two different molecules of interest on at least one molecular marker.

The method of the invention may be implemented by use of a molecular imaging method selected from MRI imaging, PET imaging, CT imaging, IF, ISH, IHC and mass spectrometry or cytometry imaging. The method of the invention is particularly suited to mass spectrometry imaging, such as MALDI, DESI, LESA, LA-ICP-MS and SIMS, for detecting the presence of the molecule of interest within the biological sample and thereby mapping said biological sample based on said detection.

The measure of the quantity or intensity of biological marker in the selected ROIs may be performed with a molecular imaging method or by other bioanalysis techniques (HPLC, LC-MS/MS, GC/MS, Magnetic bead multiplex immunoassay, ELISA).

In a particular embedment, the method comprises a step consisting on establishing a dose effect curve between one or different molecules of interest and the molecular marker, wherein each intensity or quantity of one or more molecules of interest in a ROI is represented according to the molecular marker intensity or quantity in the same ROI.

In a particular embodiment, the ROI is extracted from the biological sample by laser capture microdissection. It is then possible to cultivate the ROI in a culture medium before performing the bioanalysis, for instance to evaluate an effect of the molecule of interest by comparing gene or transcripts expression, lipidomics, peptidomics, proteomics and/or metabolics changes between the first and the second ROI.

In a particular embodiment, the molecule of interest is a therapeutic antibody, and wherein the presence of said therapeutic antibody is detected by contacting the biological sample with a marked antibody anti-therapeutic antibody.

### BRIEF DESCRIPTION OF THE DRAWINGS

FIG. 1: Molecular map of a dosed biological sample that has been exposed to a drug (FIG. 1A) and segmentation of said molecular map into two ROI wherein the drug has low intensity (on the left in FIG. 1B) and wherein the drug has high intensity (on the right in FIG. 1B). The segmentation allows discriminating between areas impacted differently by the drug. The two ROIs show different concentrations of a biomarker associated to the drug (FIG. 1C), which may be correlated to the difference of concentrations for the drug, conversely to a global method as LCMS (FIG. 1D).

FIG. 2: Molecular map of a tissue sample which has not been exposed to the drug (FIG. 2A—CTRL: Control) and molecular map of a tissue sample which has been exposed to the drug (FIG. 2B—TRT: Treated). The whole first molecular map is used as a ROI whereas the second molecular map is segmented to select a ROI with a high concentration for the drug. Both tissue samples have the same origin (tumor tissue). The ROI from the control shows high concentration for a biomarker associated to an absence of the drug, whereas the ROI from the treated sample shows low concentration for said biomarker and high concentration for the drug (FIG. 2C).

FIG. 3: Molecular map of a tissue sample which has not been exposed to the drug and molecular maps of tissue samples from three biological triplicates (FIG. 3A—CTRL, TRT Tumor 1, TRT Tumor 2 and TRT Tumor 3). The whole molecular maps are used as ROI for the corresponding samples. All tissue samples have the same origin (tumor tissue). The ROI from the control shows high concentration for a biomarker associated to absence of the drug, whereas the ROI from the treated samples show lower concentrations for said biomarker that may be correlated to their respective concentrations for the drug (FIG. 3B).

FIG. 4: Molecular map of a tissue sample which has been exposed to a first drug (FIG. 4A—Drug A), molecular map of a tissue sample which has been exposed to a second drug (FIG. 4B—Drug B) and molecular map of a same biomarker (Biomarker, FIGS. 4A and 4B). The efficacy of drug A and drug B is evaluated by reference to the concentration of the biomarker within the two or three ROIs previously identified relatively to the drug's concentration (FIGS. 4C and 4D).

FIG. 5: High level image segmentation allows obtaining multi-segments map (pixels map). Each pixel contains the drug (Drug A or B) and biomarker related information in μg/g tissue going from C1 to C11 concentrations (FIG. 5A). Drug biological effect is thus obtained per segment (pixel) that corresponds to different concentrations or intensity levels of the biomarker based on different concentrations or intensity levels of the drug (FIG. 5B). Different curves could be then drawn which would give a drug dose effect in a single tissue. ED50: Median Effective Dose, i.e., the dose required to achieve 50% of the desired response.

FIG. 6: Molecular map of a tissue sample which has been exposed to a drug (FIG. 6A), automatic segmentation of the molecular map and identification of the ROI (FIG. 6B). Schematic representation of the ROI extraction by laser capture microdissection (FIG. 6C).

FIG. 7: Epacadostat (EPA) calibration curve/QMSI (A), EPA calibration curve/LC-MSMS (B), EPA quantification by QMSI and LC-MS/MS (C).

FIG. 8: Epacadostat drug detection, quantification and histological localization. CT26 control and treated tumor sections of 10 μm thickness were analyzed in duplicate by MALDI MSI after 1,5-DAN matrix deposition. Epacadostat drug histological localization is shown present in the treated and absent in the control tumors. EPA absolute quantification in μg/g was performed and showed in the bottom table for both duplicate (1 and 2) (A). LC-MS/MS and QMSI EPA quantification were compared in treated CT26 tumors showing 17% of variability between both techniques (B). Scale colors and bars are shown for all sections.

FIG. 9: Target exposure analysis. Three regions of interest (1, 2 and 3) were selected showing different EPA quantities. ROI 1 and 2 represented 38% and 62% of the entire tumor surface. Specific EPA localization was found showing 61% concentrated in the 38% of the surface and 39% over the 62% left tumor surface (A). Semi-quantitative IDO1 immunostaining was performed on serial tissue section and inserts 1 and 2 show different expression levels of IDO1 enzyme over both regions (B). All of these overlays were performed using ImaBiotech's multimodal platform: Multimaging software.

FIG. 10: Histological localization and absolute quantification. EPA, Trp and Kyn histological localization were shown on both control and treated CT26 tumor sections (A). QMSI and LC-MS/MS analysis of Trp and Kyn were then performed and Kyn/Trp ratios were then obtained from control and treated CT26 tumors. A decrease of six times the level of Kyn/Trp ratio was noticed using both QMSI and LC-MS/MS analysis (B).

FIG. 11: Normalized Trp and Kyn calibration curves: x=concentration in mg/g; y=intensity (FIG. 11A). Kyn/Trp ratio for Plasma and blood samples (FIG. 11B).

FIG. 12: Exposure to response and response efficacy analysis. Regional pharmacological effect of EPA and response efficacy were highlighted following Kyn quantity in the three segmented ROIs; wherein 15% of Kyn is concentrated in 38% of the entire tumor (ROI 1) and 85% in ROI 2 (A). Relative intensity of EPA, Kyn and Lactate were also extracted from the molecular images (B) in order to show the response efficacy when EPA was highly or lowly present (C). Regional segmentation, relative and absolute quantifications were performed using ImaBiotech's multimodal platform: Multimaging software.

### DETAILED DESCRIPTION OF THE INVENTION

The method of the invention is based on the segmentation of a biological sample based on the concentration of a molecule of interest and the comparison of molecular changes associated to the presence of the molecule of interest within different parts/segments of a same biological sample or between different biological samples, which may comprise different amounts of said molecule of interest and/or different molecules of interest. More particularly, according to the present invention, imaging technology is used to detect and localize a compound of interest in a biological sample, which was previously exposed to said compound. The biological sample is then segmented, based on the intensity of said compound within the sample, and molecular changes are analyzed by comparing the samples segments. The method allows to identify molecules (i.e., biomarkers) that increase or decrease, even at cellular level, due to the presence of the target molecule and/or due to different doses of said target molecule. The method of the invention may also allow to compare the molecular impact of a compound of interest between two or more regions of a same biological sample, in order to determine with accuracy the therapeutic and/or prophylactic and/or toxic impact of said compound within a target biological sample. It is then possible to identify new potential drugs together with their dose effect and eventually associated biomarkers.

According to the invention, imaging technology is used to localize the molecule of interest, then image segmentation is used to analyze the molecular images of the biological sample based on the relative or absolute quantity of a molecule of interest throughout said biological sample. The analyze of the selected segments allows to determine the molecular changes that may be attributed to the presence or particular dose(s) of the molecule of interest and thereby selecting valuable biomarkers for said molecule of interest and/or screening for valuable drugs, etc.

Conversely to methods of the prior art, the method of the present invention may provide molecular efficacy information correlated to a molecule of interest within different part of a biological sample, including between different cells of a same biological tissue. It is thus possible to evaluate the real impact of a molecule within a target biological sample. Active compound/Biological marker ratio per image pixel can be calculated, which would give a drug dose effect in a single tissue, assuming that one molecular map of the biological sample represents a concentration range of the compound.

### Preparation of the Biological Sample

According to the method of the present invention, it is possible to analyze all kind of biological samples which has been previously exposed to a molecule of interest. More generally, the biological sample encompasses all system that express cell activities. This includes for instance cellular culture, 3D in vitro models such as spheroids or mini-organs, in vivo system like animal models, plants, xenograft tissues, tumors and animal biopsies, biological fluids, etc. Advantageously, the biological sample is selected from a tissue sample (e.g., a biopsy), a biological fluid sample (e.g., as urine sample, plasma sample, cerebrospinal fluid) and a cell suspension.

In the context of the invention, the term «tissue» refers to a set of functional grouped cells. The target tissue can be a set of similar or different cells with same origins, an organ, a part of an organ, a specific region of an organ with, optionally, multi-cells assemblies. For example, the target tissue can be a tumor localized within an organ. In another embodiment, the tissue sample is a plant tissue sample. More generally, a tissue sample refers to any kind of tissue of biological origin in a form that may be analyzed by an imaging method.

A biological fluid encompasses all fluid from biological origin that comprises cells and/or that has been obtained from an animal or a plant.

In the context of the invention, “exposed to” a molecule of interest means that the biological sample has been contacted with said molecule. In the context of the invention, a biological sample that has been exposed to the target molecule is referred as “dosed or treated biological sample”. Conversely, a “control sample” refers to a biological sample that has not been exposed to the target molecule. According to the invention, a control sample has same biological origin than the dosed sample to which it is compared. For instance, if the dosed tissue sample consists on a tissue section of a liver sampled in a mouse that has been exposed to the target molecule, the control sample will consist on a tissue section of a liver sampled in a mouse that has not been exposed to the target molecule.

In a particular embodiment, the molecule of interest has been previously administered to an animal model, preferably a mammal, including human and non-human mammal, and said animal has been sampled previously to the embodiment of the method. In a particular embodiment, the animal model is a non-human mammal. In another particular embodiment, the animal model is a human mammal.

Depending on the desired study, the animal model can change. The skilled person knows which animal model is well adapted depending on target tissue, molecule of interest, biological properties to evaluate, etc. For example, in the case of pre-clinical trials on a candidate molecule requiring the sacrifice of the animal, non-human mammals such as rodents (mice, rats, rabbits, hamster, etc.) are preferentially used. Others non-human mammals can be used, especially monkeys, dogs, etc. In certain cases, wherein the biological sample can be sampled without detrimental side effects for the animal, it is possible to use human as animal model (e.g., for phases 1-4 of clinical trial). It is also possible to use others animal models such as fishes, insects, for instance to study the impact of a molecule on the environment or a particular ecologic medium.

According to the invention and in a general term, all administration route of the target molecule can be used, such as enteral route (i.e. drug administration by the digestion process of a gastrointestinal tract) or parenteral route (i.e. other route of administration than by the gastrointestinal tract). For example, the molecule can be administrated by different routes such as epicutaneous, epidural, intra-arterial, intravenous, subcutaneous (with a specific localization), intra-cardiac, intra-cavernous inject, intra-cerebral, intradermal, intramuscular, intra-osseous infusion, intra-peritoneal, intra-thecal, intra-vesical, intra-vitreal, nasal, oral, rectal, intra-vaginal, or by topical application.

The administration route can be chosen depending on the molecule of interest, the tissue targeted by the method, etc. The method of the invention can also permit to select the most adapted route of administration. Indeed, the method of the invention allows evaluating the ability of a molecule of interest to cross a biological barrier to reach the target tissue.

According to the invention, the method is preferably performed ex-vivo and/or in-vitro. It is also possible in some case to perform in-vivo analysis on the living whole animal.

In a particular embodiment, the method of the invention is an ex-vivo analysis, for example on a tissue section. In that case, the tissue sample is sampled at a given time post administration (t1). The sampling can be a biopsy, especially when it is a. human mammal. According to an embodiment, after administration of the molecule of interest to a non-human animal, said non-human animal is sacrificed and the tissue sample of interest is sampled.

In a particular embodiment, the tissue sample is obtained from fresh tissue, frozen tissue, or fixed/embedded tissue, for example with paraffin. All means suitable for obtaining thin tissue sections, as a few micrometers thick, can be used.

If necessary, the tissue section can receive a pretreatment, especially depending on molecules to be detected, the analytical technique, etc. Thus, it is possible to use chemical or biochemical agents on tissue sections to optimize the detection of the molecule of interest and biomarkers. For example, it is possible to use solvents and or detergents to permit the detection of define classes of molecule or improve the direct extraction of molecules from tissue. As well, it is possible to use specific enzymes capable of cleaving peptides or proteins, in order to target for example, digest fragments which have the same localization and/or amount on tissue as the parent molecule. It is also possible to add some analytes on-tissue or in-tissue for a chemical derivatization of the molecule of interest, since it addresses some sensitivity issues allowing increasing the on-tissue distribution and quantification studies. It is also possible to perform antibody labeling (coupled or not with a tag), on tissue sections, or to use fluorescence labeled molecules or radioactivity to allow the detection of the molecule of interest and biomarkers.

It is also possible to change the animal model used and/or the target tissue and/or the tissue section in order to modify their abilities to bind or incorporate the molecule of interest. Thus, this treatment can include a chemical or biological modification of animal model and/or target tissue and/or tissue section which permits to increase or inhibit the penetration or targeting ability of a molecule of interest for a given target tissue. This treatment can be performed previously, subsequently or simultaneously to the administration of the molecule of interest. For example, in the case of molecules that must cross the blood brain barrier (BBB), there is some efflux transporters in the barrier which are able to eject the molecules crossing the BBB. The effect of these transporters can be modulated (decreased or suppressed) using inhibitors or genetic modification, as a “knock-out”, on the gene or the gene expression of said transporters.

In the case of a liquid sample, it is possible to dry it on a surface in order to produce the MS image of the dried sample, and then to characterize this sample with MSI.

If mass spectrometry imaging requiring a matrix is used to study biological sample, and notably, MALDI or ME-SIMS (Matrix Enhanced Secondary Ion Mass Spectrometry), the said matrix is advantageously adapted to the molecule of interest. For instance, the choice can take into account the mass range covered. The skilled person knows, from existing liquid or solid matrices, which one can be used depending on studied molecules and/or target tissue. Similarly, all deposition method of the matrix can be used, especially manual spraying, automatic spraying, sublimation, sieving and micro spotting.

In another embodiment, the molecule of interest is contacted in vitro or ex vivo with a biological sample, such as a tissue sample, a biological fluid, a cell suspension, etc. For instance, the biological sample is a cell suspension and the molecule of interest is added to the suspension before to perform the analysis. Alternatively, the biological sample can be deposited onto a support (and in case of fluid sample, it may be optionally dried) and the molecule of interest is sprayed onto the sample before to perform the analysis. Detection of the molecule of interest

The biological sample is analyzed in order to detect the presence and optionally the concentration of the molecule of interest within said biological sample.

This step can be performed using any technique allowing the accurate identification and visualization, in vivo, in vitro or ex vivo, of molecules within a biological sample.

Notably, in the case of in vivo analysis, it is possible to use a tomographic technique such as the magnetic resonance imaging (Mill), the autoradiography, the positron emission tomography (PET), the mono-photon emission tomography, CT imaging, etc.

In the case of in vitro/ex vivo analysis, it is possible to use IF, IHC, ISH and mass spectrometry imaging (MSI). Techniques such as MALDI imaging (Matrix-Assisted Laser Desorption/Ionization), LDI (Laser Desorption/Ionization), DESI (Desorption by Electrospray), LESA (Liquid Extraction Surface Analysis), LAESI (Laser Ablation Electrospray Ionization), DART (Direct Analysis in Real Time), SIMS (Secondary ion mass spectrometry) JEDI (Jet Desorption Electrospray Ionization), optionally in combination with different kinds of mass analyzer as TOF (Time of flight), Orbitrap, FTICR (Fourier Transform Ion Cyclotron Resonance), quadruple (simple or triple), etc., can be used. Preferably, the mass spectrometry imaging is selected from MALDI, DESI, ICP-MS and SIMS.

Experimental parameters such as mass range, laser fluency, laser focus etc., are fixed to optimize target detection in terms of intensity, sensitivity and resolution. Thus, the acquisition of mass spectra is performed to obtain a signal. From the mass spectrum, it is possible to have access to useful data for target molecules study. For data treatment, different spectral characteristic can be used as the peak intensity on mass spectrum, the signal to noise ratio (S/N), the peak area, etc.

As a general rule, all techniques allowing the visualization of molecule within a biological sample can be used.

### Selection of Region(s) of Interest (ROI)

The analysis of the biological sample provides a molecular map of the biological sample at least for the molecule of interest. That is to say that an image of the molecule of interest throughout the biological sample is obtained. The image allows visualizing directly the distribution and the concentration of the molecule of interest within the biological sample. Thus, the absence and/or presence of the molecule within different regions of the biological sample may be visualized but also the difference of concentrations between said different regions, assuming that one molecular map of the biological sample represents a range of concentrations. The molecular map of the biological sample is then segmented into different regions.

The segmentation of the biological sample can lead to regions of various dimensions, from a pixel to a segment (comprising a plurality of pixels). The segmentation may be performed manually or automatically, in order to obtain different regions characterized by the presence/absence/intensity/quantity of the molecule of interest (i.e., spectral information of the molecule of interest). For instance, the segmentation may be performed automatically with image segmentation techniques selected from region-based segmentation, edge detection segmentation, segmentation based on clustering, segmentation based on weakly-supervised learning in CNN, etc. Then, regions of interest (ROI) can be selected.

The size of the ROIs can be different from each other. A ROI can be an average of many image pixels or segments. Each image pixel or segment can also represent a ROI having different intensity or concentrations for the molecule of interest.

In a particular embodiment, the biological sample is a tissue section that has been analyzed by MSI. The image obtained shows the overlay distribution of the molecule of interest. The segmentation is thus performed based on the peak intensity, peak area or signal to noise ratio of the molecule of interest.

For instance, the molecule of interest is a candidate molecule and the dosed biological sample has been previously obtained by sampling in an animal that has been previously administered with the candidate molecule.

In an embodiment, the second ROI is selected from the segmentation map of the dosed biological sample, said second ROI being physically different from the first ROI and having a second intensity for the molecule of interest. Advantageously, the first ROI selected exhibits the most important signal (and thereby the most important concentration) and the second ROI selected does not exhibit any signal for the molecule of interest (i.e., is deprived of the molecule of interest). Alternatively, the second ROI of the dosed biological sample exhibits low signal for the molecule of interest (and thereby low concentration).

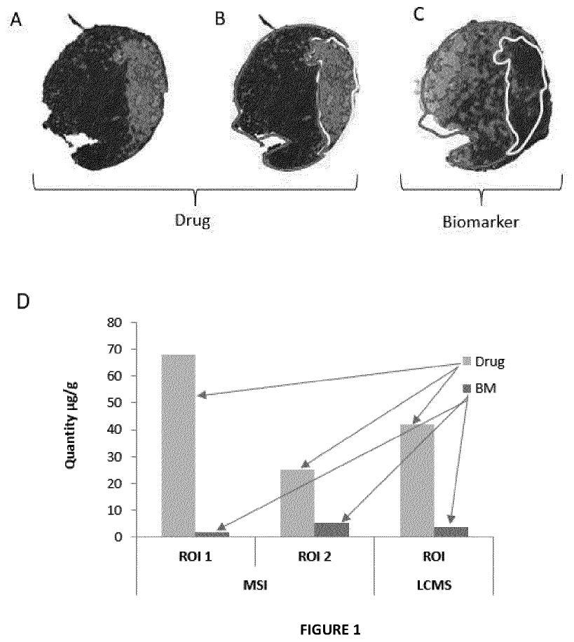

FIG. 1 shows the molecular map of a tissue section for a drug obtained with MSI. The tissue section has been sampled in an animal that has been previously administered with the drug. The molecular map (FIG. 1A) is based on the intensities associated with the molecule. From the spectral data visualized on the tissue section, it is possible to delimit two ROIs within the same tissue section (FIG. 1B). The first ROI corresponds to the region of the tissue sample that exhibits a high signal for the molecule, whereas the second ROI corresponds to the region of the tissue sample that exhibits a low signal for the molecule.

In another embodiment, the second ROI is selected from a second biological sample which has not been previously exposed to the molecule of interest, said second biological sample being from same origin as the dosed biological sample (i.e., control sample).

In the context of the invention, “same origin” means that the biological samples are sampled on same animal models (e.g., two mice) and the samples are identical (e.g., both biopsies from same tumor cell lineage tissues, both liquid samples of urine, etc.).

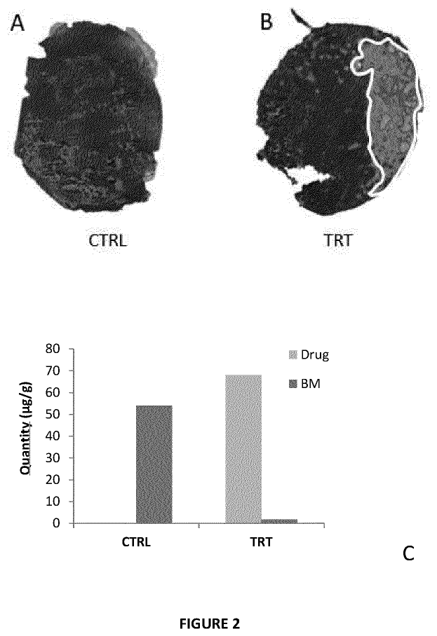

FIG. 2 shows the molecular maps of two tissue sections of same origin (tumor tissues) for a drug obtained with MSI. The first tissue section (FIG. 2A) has been sampled in an animal which has not been contacted with the drug (control, CTRL). The second tissue section (FIG. 2B) has been sampled in an animal that has been previously administered with the drug (treated, TRT). The first tissue section as a whole is used as a first ROI. The second ROI is selected in the second tissue section, and corresponds to the region of the second tissue section wherein the molecule is highly detected.

In another embodiment, the second ROI is selected from a second biological sample, which has been previously exposed to the molecule of interest at a dose concentration different from the dose concentration for the dosed biological sample, said second biological sample being from same biological origin as the dosed biological sample.

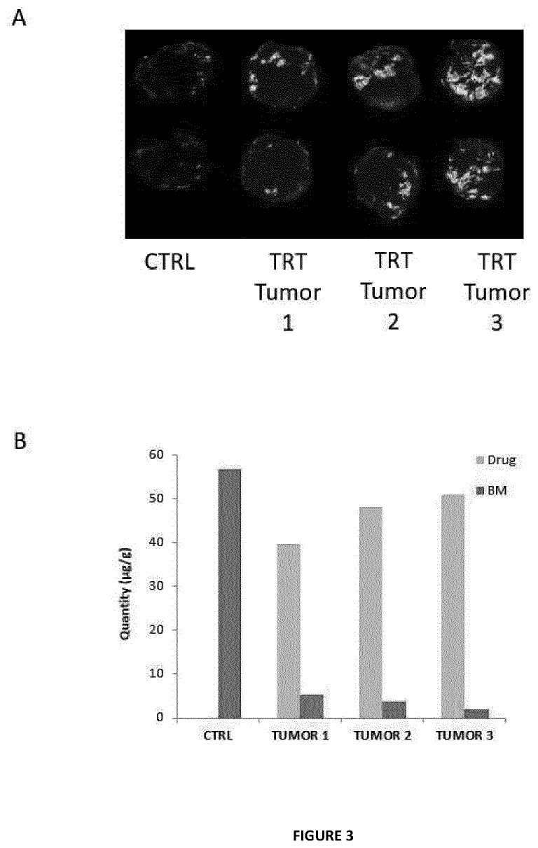

FIG. 3 shows the molecular maps of four tissue sections of same origin (tumor tissues) for a drug obtained with MSI. The first tissue section has been sampled in an animal which has not been contacted with the drug (control, CTRL). All the other tissue sections have been sampled in animals that have been previously administered with the same concentration of the drug (TRT1, TRT2, TRT3), but wherein the concentration within the samples are different. The ROI for each biological sample is the whole corresponding tissue section. Of course, it would be possible to limit the ROI to a particular region of each tissue section, such as the ROI which exhibits the most important concentration for the drug.

In another embodiment, the second ROI is selected from a second biological sample which has been previously exposed to a same or a second molecule of interest, said second biological sample being from same origin as the dosed biological sample.

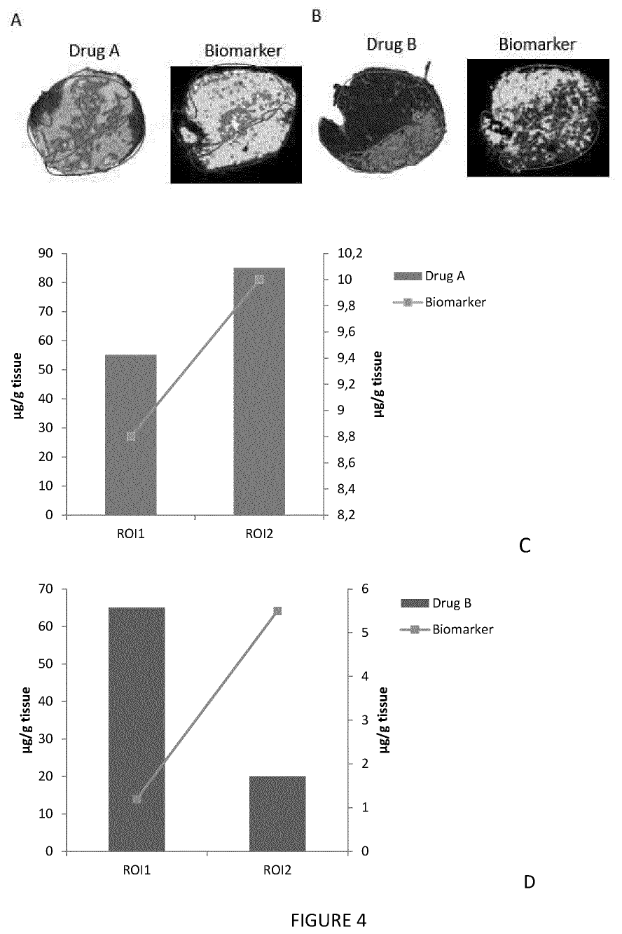

FIG. 4 shows the molecular maps of two tissue sections of same origin (tumor tissue) for two different drugs (FIG. 4A: Drug A; FIG. 4B: Drug B) obtained with MSI. The tissue sections have been sampled in animals that have been previously administered with either drug A and drug B. The ROI1 and ROI2 were manually segmented on each biological sample. In order to know the drug effect that drug A and B could have on the Biomarker, its intensity/concentration was then calculated in each ROI (1 and 2) and drawn for each corresponding ROI (FIGS. 4C and 4D).

In a particular embodiment, the method of the invention may be used for establishing a dose effect curve between one or more molecules of interest and molecular marker(s), wherein each intensity or quantity of a molecule of interest in a ROI (a single pixel or several pixels) is represented according to the molecular marker intensity or quantity in the same ROI.

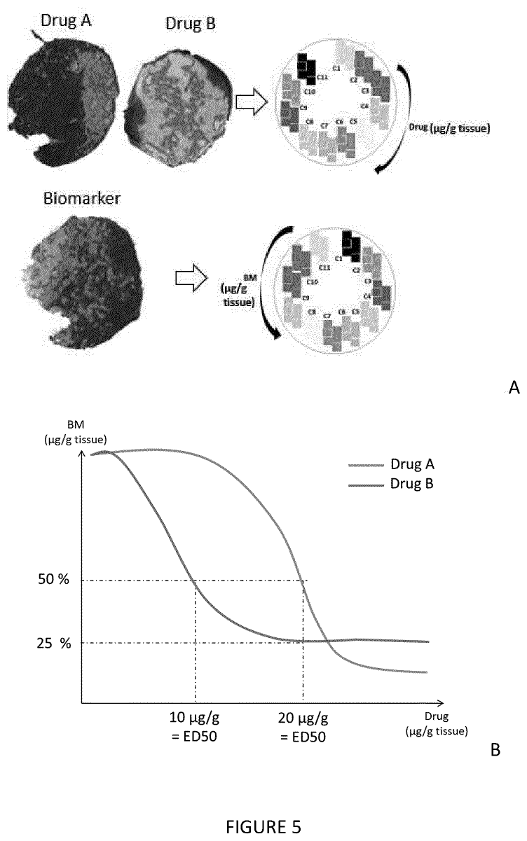

FIG. 5 shows a molecular map of the biological sample that represents a concentration range of the compound of interest. A higher level of image segmentation allows obtaining multi-segments map (pixels map). Each pixel contains the drug and biomarker related information in μg/g tissue going from C1 to C11 concentrations (FIG. 5A). Drug biological effect is thus obtained per segment (pixel) that corresponds to different concentrations or intensity levels of the biomarker based on different concentrations or intensity levels of the drug (FIG. 5B). Different curves could be then obtained, which would give a drug dose effect in a single tissue. ED50: Median Effective Dose, i.e., the dose required to achieve 50% of the desired response.

### Biomarkers Analysis

A molecular analysis is performed on each selected ROI, in order to measure the quantity or intensity of one or more biological markers within the ROI of the corresponding biological samples.

The molecular analysis leads to an understanding of the composition of the ROIs at a molecular level and/or to the detection and optionally quantification of one or more biological markers. Advantageously, all ROIs are analyzed with the same method.

According to an embodiment, the measure of the quantity or intensity of the biological marker in a ROI is performed with a molecular imaging method. Preferably the molecular imaging method selected from MSI, ISH, IF, IHC, MRI, imaging mass cytometry, CT and PET imaging.

In a particular embodiment, both ROI are analyzed by MSI. MSI refers to ICP-MS, LA-ICPMS, LAESI, MALDI, DESI, SIMS, LESA and similar surface extraction strategy, SIMS, etc. Basically, mass spectrometry imaging (MSI) is able to simultaneously record the distribution of hundreds of biomolecules directly from tissue or fluid sample, without labeling and without prior knowledge. MSI based on matrix assisted laser desorption/ionization (MALDI) applied with different tissue/fluid preparation procedures can be used to analyze proteins, peptides, glycans, lipids, metabolites and drugs.

According to another embodiment, the measure of the quantity or intensity of the biological marker in a ROI is performed by bioanalysis. Preferably, the bioanalysis is selected from mass spectrometry, electrophoresis, ligand binding assay, nuclear magnetic resonance, microdialysis and chromatography.

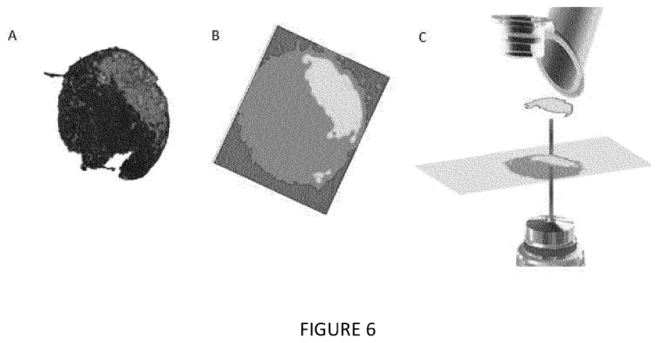

In a particular embodiment, the ROIs are extracted from the biological sample before to analyze the biological marker(s). In the context of the invention, “extraction of a ROI” means that the ROI is physically separated from the rest of the sample. Physical extraction may be performed by microdissection, manually or by laser. FIG. 6C illustrates a laser capture microdissection (LCM) performed on a tissue section. The ROI of interest is thereby sampled and separated from the rest of the tissue section. This extraction allows to perform the bioanalysis only on the part of the tissue section that has to be considered and to discriminate with more accuracy between different part of a same tissue section.

FIG. 6 shows a molecular map of a tissue sample which has been exposed to a drug (FIG. 6A), then a manual or an automatic segmentation of the molecular map and the identification of the ROI (FIG. 6B), followed by a ROI extraction by laser captures microdissection (FIG. 6C).

According to a particular embodiment, the extracted ROI is cultivated in a culture medium before performing the bioanalysis.

In a particular embodiment, a bioanalysis is performed on both ROIs, and an effect of the molecule of interest is evaluated by comparing gene and transcripts expression, lipidomics, peptidomics, proteomics and/or metabolics changes between the first and the second ROI (or others).

The molecular analyses of the ROIs and the comparative study of the results may lead to different understanding.

According to the invention, the method implemented for the molecular analysis may be a direct method or an indirect method (e.g., using tag molecules like antibodies or RNA sequence). For instance, the molecular analysis may be performed by use of IHC, ISH or FISH imaging, LA-ICP imaging using tagged antibodies or alone to detect metals on tissue, genomic analysis, SNIPS, LC-MS analysis of the region of interest.

In a particular embodiment, the biomarker associated to the molecule of interest is already known, and the analysis step focus on the detection and/or quantification of said biomarker. The method of the invention may be used to identify at a very fine level the target tissue. The method of the invention may also be used to determine the best dose of the expected effect of a drug within a target tissue and/or for screening drugs.

FIG. 1 illustrates an embodiment wherein a biomarker (BM) associated to a drug is already known. The molecular maps of the tumor tissue show that the drug is localized on a specific part of the tissue (FIG. 1B). The analysis of the concentration of the biomarker within the corresponding ROIs shows a decrease of the concentration of the BM in the ROI wherein the drug is concentrated, as compared to the ROI wherein the drug is almost absent (FIG. 1C). The method of the invention allows discriminating between different part of a same tissue section, whereas a global analysis with standard method (LCMS) only shows the global impact of the drug in the whole tissue section.

FIG. 4 illustrates an embodiment wherein the method of the invention is used for screening a drug. The expected effect associated to two different candidates (Drug A vs. Drug B) is analyzed (i.e., decrease of the concentration of a biomarker) according to the drug level in ROI1 and ROI2.

FIG. 5 illustrates an embodiment wherein a biomarker (BM) associated to a drug is already known. The method of the invention is implemented in order to determine the dose effect of the drug from one sample containing analytical replicates and avoiding biological bias. The analysis of the concentration of the biomarker within the different pixels shows the impact of the concentration of the drug within the tissue on the concentration of the biomarker (FIG. 5B). The method of the invention allows determining the dose of a drug required to obtain the desired effect, or even the median effective dose (the dose required to achieve 50% of the desired response). As illustrated in FIG. 5B, for 50% of efficacy, drug B is more potent than drug A. Conversely, for 25% of efficacy, drug A is more potent than drug B.

According to another embodiment, the analysis of the ROI is implemented to identify biomarker(s) associated to the molecule of interest.

Mass spectrometry delivers molecular information about the biological impact of the molecule of interest as an increase or a decrease of lipids, metabolites, peptides or proteins concentration in living systems. Other techniques (IF, IHC, ELISA, ISH, spectrophotometry or UV fluorescence) allow evaluating and quantifying peptides, proteins, RNA (Ribonucleic acids) and DNA (desoxyribonucleic acids) that are targeted using a tagged antibody or nucleic sequence that will bind to the targets. All or part of these embodiments may be combined to show the impact of different molecules of interest on a same biological sample and/or the impact of a molecule of interest at different concentrations and/or the impact of a molecule on different biological samples of interest, etc.

According to the invention, it is possible to normalize the molecular information by use of endogenous compounds with multiplex imaging technique (e.g., Mass Spectrometry Imaging) to segment the images related to the molecule of interest. This may help to minimize the variability. In this case, an external or internal standard compound is used as a reference compound.

### Applications

The examples below briefly expose different applications for the method of the invention. After administration of a drug to at least one biological sample (e.g., in vitro tests, in vivo animals), imaging technology is used to detect and localize the drug. A molecular map of the sample for the drug is obtained. The molecular map is segmented based on intensity and two regions of interest (ROI) are selected.

1—Different analytical methods are used to identify molecular changes (e.g., methods of mass spectrometry or mass spectrometry imaging to target metabolites, DNA, RNA, proteins, lipids, peptides, metals or other technique of imaging ISH, IHC, MRI, PET imaging to compare the two regions of interest). Then, the molecules that increase or decrease when the drug is localized are identified. It is possible to score the results regarding the drug and demonstrate the impact of the drug to certain cells or sub-structure of the organs.
2—The ROIs are extracted manually or automatically (with laser micro dissection instrument) and analytical methods are used to identify molecular changes. Methods of bioanalysis such as mass spectrometry or other technique like PCR are used to compare the content of the two regions of interest. It is possible to score the results regarding the drug and demonstrate the impact of the drug to certain cells or sub-structure of the organs.
3—Different analytical methods are used to identify molecular changes, such as mass spectrometry or mass spectrometry imaging to target metabolites, DNA, RNA, proteins, lipids, peptides, metals or other technique of imaging ISH, IHC, MRI, PET imaging to compare the two regions of interest. The molecules that increase or decrease when the drug is localized are identified. It is possible to score the results for untargeted compounds and to demonstrate the impact of the drug to certain cells or sub-structure of the organs.
4—Two different drugs in two biological samples are used. Two ROIs are selected, corresponding to the localization of the corresponding compounds within the corresponding biological sample. Different analytical methods are used to identify molecular changes, such as methods of proteomics, metallomics, transcriptomics, genomics and metabolomics, including lipidomics to compare the two regions of interest. Then, the molecules that increase or decrease where the drugs are localized are identified. It is possible to score the results for targeted or untargeted compounds and to evaluate the dose response (efficacy or toxicity) in the particular region where the drug is located.
5—Two different drugs in a same biological sample are used. With the same process of ROI selection and comparison with analytical tools, two ROI are selected. Different analytical methods are used to study the differences in molecular expression. It is also possible to select a ROI where both drugs are located and study their impact on molecular changes and possible synergistic effect.
6—More ROIs can be created as image pixels to determine the dose effect of a drug based on a drug range of concentrations from one sample containing analytical replicates and avoiding biological bias. The analysis of the concentration of the biomarker within the different pixels shows the impact of the concentration of the drug within the tissue on the concentration of the biomarker. The method of the invention allows determining the dose of a drug required to obtain the desired effect, or even the median effective dose (the dose required to achieve 50% of the desired response).
7—A biological sample is contacted in vitro with a drug that will penetrate into cells and will induce molecular interactions and physiological changes. Using the same process of ROI selection and comparison with analytical tools, potentially different ROIs are selected corresponding to different cells phenotypes and the corresponding responses to the drug are analyzed (mutagenesis, toxicity, etc.).

### Examples

Indoleamine-2,3-dioxygénase (IDO1) is an enzyme which converts tryptophan (Trp) into kynurenine (Kyn). Having a critical role in tumor immune escape by decreasing Trp and increasing Kyn levels in the microenvironment, IDO1 was one of the first targets for small molecules drug discovery in the field of immuno-oncology (I-O). A potent and selective IDO1 inhibitor such as Epacadostat (EPA) was shown to enhance the antitumor activity by restoring the immune system fitness.

In this study, using quantitative mass spectrometry imaging (QMSI), the aim was to quantify the metabolites and the drug, but also to reach a higher information level by their histological localization and quantification. Here, a quantitative ex vivo study is reported that allowed highlighting the Epacadostat (EPA) target exposure and pharmacological effect efficacy in specific regions of the tumor. As exposure at the site of action and to its specific target are identified as the most important factors for success in drug discovery, the objective of this study was to explore the target exposure and intra-tumor pharmacodynamics effects of an IDO1 inhibitor on the tumor metabolism.

### 1. MATERIALS AND METHODS

1.1. Chemicals and Reagents

All chemicals including 1,5-diaminonaphtalene (1,5-DAN), 2,5-dihydroxybenzoic acid (2,5-DHB), kynurenine-d4; tryptophane-d5, formic acid and trifluoroacetic acid were purchased from Sigma-Aldrich (St. Louis, Mo.). Kynurenine-¹³C was purchased from Alsachim. Methanol, acetonitrile, and Optima LC-MS water were purchased from Fisher Scientific (Waltham, Mass.). Indium tin oxide (ITO)-coated glass slides were purchased from Bruker Daltonics (Bremen, Germany).

Primary rabbit anti-mouse IgG IDO [#106134] and secondary goat anti-rabbit IgG H&L (HRP) secondary antibody [#205718], and detection system through horseradish peroxidase followed by Steady DAB/plus (brown chromogen) were all purchased from AbCam (Cambridge, UK).

1.2. Sample Collection and Tissue Preparation

Colon carcinoma CT26 cell line was subcutaneously grafted into BALB/c mice (Charles River Laboratories, France). Mice were randomized and treatment was started when tumors had an average size of 70 to 120 mm³. Mice were treated by oral gavage with the IDO1 inhibitor, Epacadostat (Syngene, India) at 100 mg/kg, and then sacrificed 2 hours later. Tumors were sampled and snap frozen in liquid nitrogen for 15 s. The samples were kept at −80° C. until use. Ten micrometers thick tissue sections were obtained using a cryostat microtome (CM-3050S, Leica, Germany) with a microtome chamber and a specimen holder chilled at −23° C. Tissue sections were thaw mounted onto ITO-coated slides for downstream MALDI imaging and serial sections on SuperFrost slide for histological and immunohistochemistry (IHC) analysis. Biological replicates in duplicate (distant serial sections) were performed for analytical reproducibility. For LC-MS/MS analysis, five tissue sections of 10 μm thickness were harvested to perform calibration curves and quantitation of EPA, Kyn and Trp. All animal experiments were compliant with the 2010/63/UE European Directive on Laboratory Animal Welfare and were approved by an Ethical Committee.

1.3. Qualitative and Quantitative MALDI Analyses of Epacadostat Drug

The EPA calibration curve was measured following two steps (large and then restricted ranges). Then, low limit of detection (LOD), low limit of quantitation (LLOQ) and limit of linearity parameters were defined for the calibration curve that has been performed using at least 10 spotted concentrations on 10 μm thickness control tumor tissue sections. The concentration range was from 125 to 1 pmol for EPA, which was validated as a linear regression model. On the same ITO slide, calibration curve and tissue sections of interest (1 control and 3 treated in duplicates) were deposited and analyzed. Then, a uniform layer of filtrated 1,5-diaminonaphtalene (1,5-DAN) prepared at 10 mg/mL with 50/50 ACN/H₂O matrix was deposited onto the tumor tissue sections using an HTX-TM sprayer device (HTX Technologies, LLC, Carrboro, N.C.). MALDI MSI analyses were performed using 7T MALDI-FTICR (SolariX XR, Bruker Daltonics, Bremen, Germany) with a SmartBeam II laser. MSI data for Epacadostat were recorded in CASI negative ion mode (m/z range 436.0+/−30) at 120 μm of spatial resolution using an online calibration. Data acquisition was performed using the Flex software (FtmsControl 2.1.0) from Bruker Daltonics. Then, Multimaging software (ImaBiotech SAS) was used to obtain the calculated equation and to extract the different quantities in pmol/mm². A quantity conversion to μg/g of tissue was then obtained in created regions of interest (ROIs).

1.4. Qualitative and Quantitative MALDI Analyses of Trp and Kyn

As Kyn was not detectable and Trp was slightly seen using classical method in CT26 tumor models, a derivatization strategy was necessary. The derivatization reaction amounts to adding the C₁₁H₁₆N+ (as X) unit to the neutral analyte. The agent was applied using an automatic sprayer (Suncollect, Sunchrom) and left during 30 min for incubation at room temperature. Then, a uniform layer of 2,5-dihydroxybenzoic acid (2,5-DHB) prepared at 40 mg/mL with 50/50 MeOH/H₂O+1% TFA matrix mixed with Kyn-d4-derivatized internal standard at 0.5 μM was sprayed over the tumor tissue sections and the calibration curves using an HTX-TM sprayer device (HTX Technologies, LLC, Carrboro, N.C.). Using isotopic modified compounds, tissue suppression effect should allow getting the same suppression effect if present within the tissue. Note that deuterated standards (Trp-d5 and Kyn-¹³C) were used to perform the calibration curve to avoid the internal interference with the endogenous compound. Then, MALDI MSI analyses were performed using 7T MALDI-FTICR (SolariX XR, Bruker Daltonics, Bremen, Germany) with a SmartBeam II laser. MSI data for kynurenine and tryptophan was recorded in positive ion mode (CASI, m/z range 350.0+/−150) at 120 μm of spatial resolution using an online calibration. Data acquisition was performed using the Flex software (FtmsControl 2.1.0) from Bruker Daltonics.

1.5. MALDI-FTICR Imaging of Other Metabolites

For MALDI MSI of other metabolites, a uniform layer of filtrated 1,5-diaminonaphtalene (1,5-DAN) prepared at 10 mg/mL with 50/50 ACN/H₂O matrix was deposited onto the tumor tissue sections using an HTX-TM sprayer device (HTX Technologies, LLC, Carrboro, N.C.). Then, MALDI MSI analyses were performed using 7T MALDI-FTICR (SolariX XR, Bruker Daltonics, Bremen, Germany) with a SmartBeam II laser. MSI data for metabolites were recorded in full scan negative ion mode (m/z range 100-1000) at 120 μm of spatial resolution using an online calibration. Data acquisition was performed using the Flex software (FtmsControl 2.1.0) from Bruker Daltonics.

1.6. LC-MS/MS Analysis

Calibration curve (0 to 500 nM) was prepared in water containing 5 nM of internal standard (Kyn-d4 and Trp-d5). Afterwards, between 0.5 and 2 mg of serial tumor sections was collected in methanol/water extraction solution that contains 10 nM on internal standard. An overnight stirring extraction was performed at 4° C., then a centrifugation at 3000×g, at 4° C./15 min allowed to recover the supernatant from both calibration curves of Kyn and Trp and all the sections that were used for the LC-MS/MS analysis. For tumors, a dilution at ½ in water was performed prior analysis. A total of 5 μL was injected into the LC-MS/MS system. No additional filtration step was necessary. The LC-MS/MS system consisted of an ultra-high-performance liquid chromatography-focused+LC system composed of RS column compartment, RS pump, and RS autosampler (Dionex Ultimate 3000, Thermo Fisher Scientific) that contains a Waters column (Cortecs C18 75×3 mm, particle size=2.70 coupled to a TSQ Quantiva Thermo Scientific (Thermo Fisher Scientific). Data acquisition and processing were carried out using TSQ Quantiva software version 2.0 1292 and Xcalibur 3.0 software.

1.7. MSI Data Processing and Analysis

1.2 software (ImaBiotech SAS). This multimodal imaging platform combines quantitative mass spectrometry imaging (QMSI) and microscopy platform with statistical analysis for the understanding of the Omics information at cellular levels. MSI data were acquired from each tissue section as well as matrix control areas adjacent to the tissue sections to check for analyte dispersion/delocalization during sample preparation. Therefore, the ROIs related to EPA and/or Kyn presence were given by an image segmentation algorithm. First, the algorithm divided the sample in different classes based on a molecular signal threshold (2 classes or ROIs in this study case for both EPA and Kyn). Then, the algorithm smoothed the two ROIs to transform them into connected spaces. Finally, an exposure score was calculated using the Multimaging software using the formula:

$$\frac{Concentration ( ROI ) * {N}_{ROI}}{Concentration ( Total ) * {N}_{Total}}$$

with N_{ROI} the number of pixels inside the ROI and N_{Total} the number of pixels inside the entire sample.

1.8. Immunohistochemistry Analyses, Digital Scan Image, and High-Definition Overlay

Serial sections were stained for IDO1 histological localization using rabbit polyclonal antibodies purchased from Abcam (Cambridge, UK) and adapted to fresh frozen tissue sections. Sections were first exposed to 0.5% Triton-X for 15 min at room temperature and washed with phosphate-buffered saline prior the addition of the primary anti-IDO1 antibody (1:50 dilution) and processed with goat anti-rabbit IgG H&L (HRP) secondary antibody (1:2000). The detection system was through horseradish peroxidase followed by steady DAB/plus (brown chromogen).

After MSI data acquisition, any residual matrix was removed with a 100% methanol wash, and the tissue samples were then stained with hematoxylin and eosin (H&E) solution. High-resolution histological images of H&E stain or IHC were then recorded using a digital slide scanner (3D Histech Pannoramic) then loaded in Multimaging technology to perform the high definition overlays with convoluted molecular images.

### 2. RESULTS

2.1. Epacadostat Drug Detection, Quantification and Histological Localization

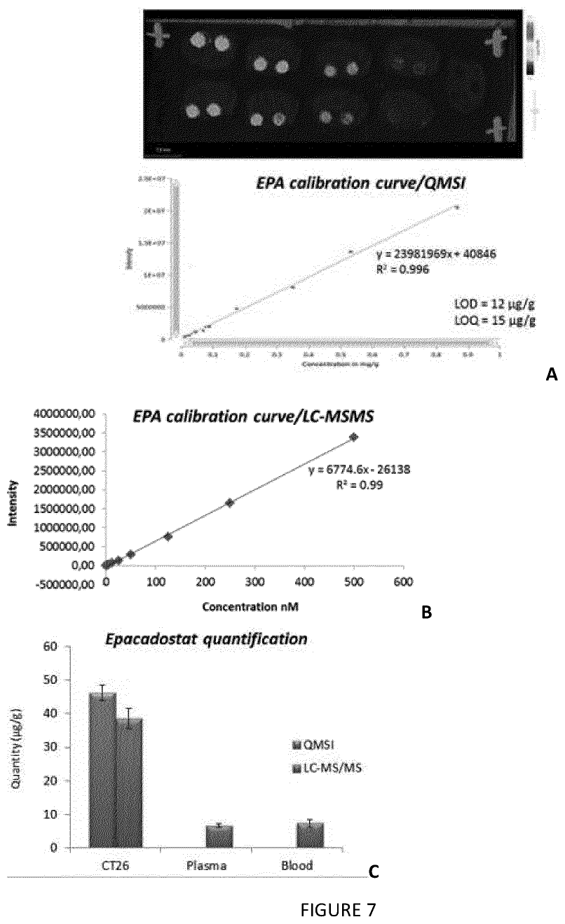

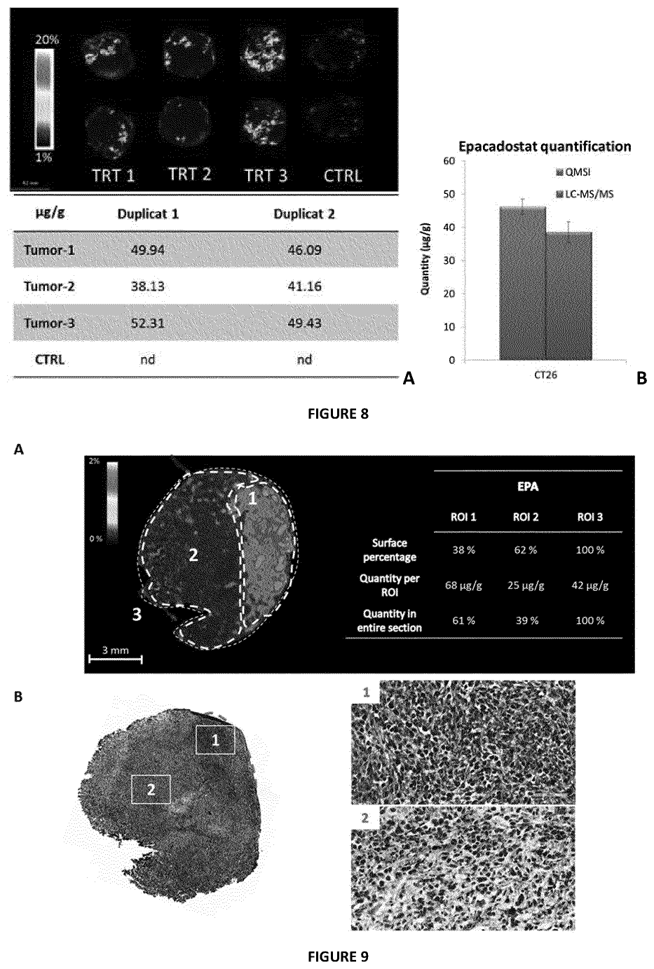

QMSI and LC-MS/MS analysis were performed to quantify the absolute Epacadostat level in CT26 tissue, plasma and blood samples. First, sample preparation and development method were carried out for EPA QMSI analysis. A calibration curve was spotted on control CT26 tissue sections, allowing getting a calibration curve (R²=0.996) showing a linearity range from 12 to 870 μg/g with a Limit of Detection (LOD) at 12 μg/g and a Lower Limit of Quantification (LLOQ) at 15 μg/g (FIG. 7A). Then, molecular images showing the histological localization of the first EPA isotope (m/z 435.9844) were showed in one control and three treated sections each in duplicate. QMSI was then performed using the obtained calibration curve giving quantities between (38 and 53 μg/g for the three biological triplicates) (FIG. 8A). LC-MS/MS quantification was also performed on serial CT26 tissue sections. FIG. 7B showed the EPA calibration curve that was obtained in nM. The average EPA quantity was at 38.5 μg/g vs 46 μg/g when using LC-MS/MS vs QMSI, respectively (FIG. 8B), showing a variation of 17% between both techniques. EPA quantification was also performed on plasma and blood (FIG. 8C).

2.2. Target Exposure Analysis

For target exposure analysis, first an automatic segmentation of EPA signal highlighted two ROIs (1 and 2). Then, EPA absolute quantification was assessed (using QMSI) and automatically extracted from the three regions (ROI 1 for high EPA, ROI 2 for low EPA and ROI 3 for the entire treated tumor). The results showed a quantity of 68 μg/g in ROI 1, 25 μg/g in ROI 2 and 42 μg/g in the entire section (ROI 3); which means that almost 61% of EPA signal (26 μg/g) was concentrated within 38% (ROI 1) of the entire tissue section and 39% (15 μg/g) was in the 62% left tumor region (ROI 2) (FIG. 9A). Thereafter, the intensity of IDO1 enzyme expression was highlighted by immunostaining. Thus, the semi quantitative analysis allowed to distinguish two regions showing different levels of IDO1 expression moving from the highest (1) to the lowest one (2) (FIG. 9B). A positive correlation between IDO1 expression level (semi-quantitative assessment by histology) and the EPA levels was interestingly also noticed.

2.3. Pharmacodynamics Study of Kyn and Trp Metabolites

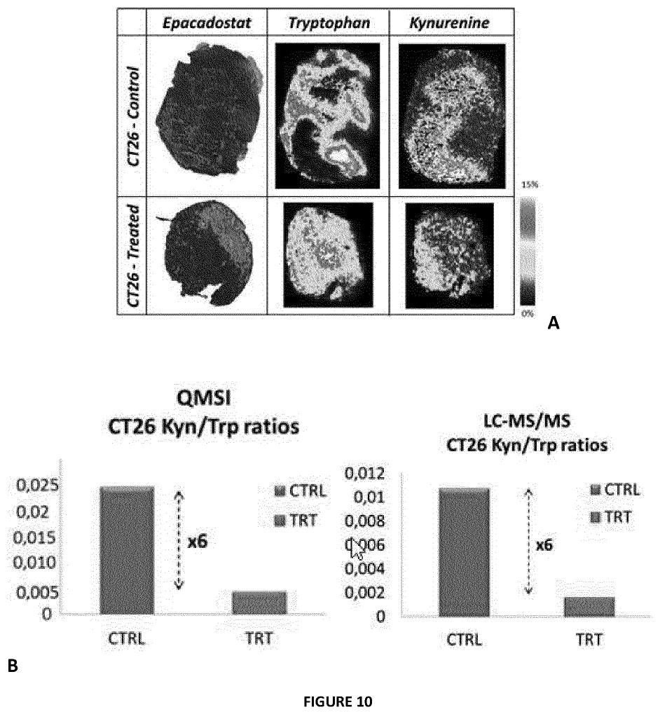

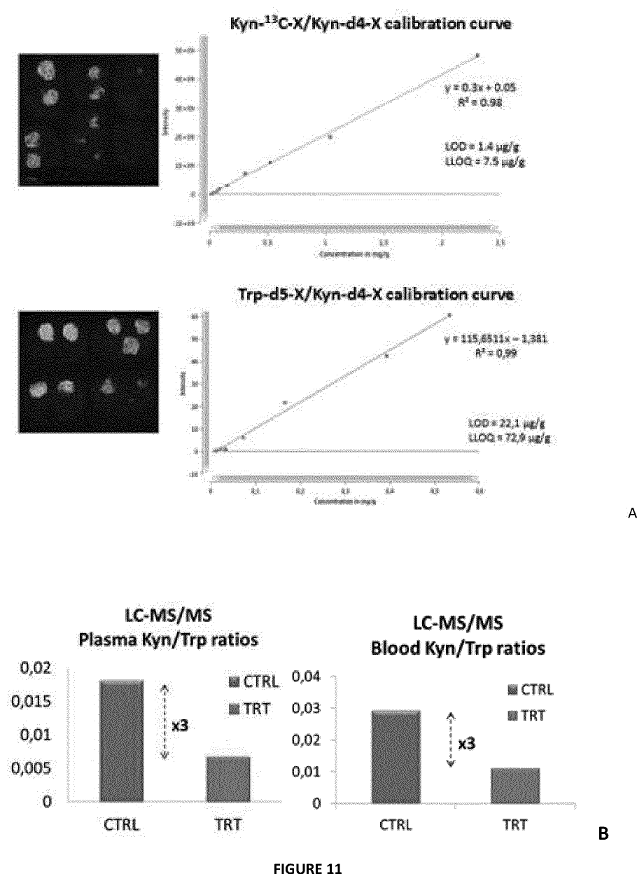

Regarding the endogenous metabolites, the optimized derivatization step allowed both Trp and Kyn detection and quantification when improving their sensitivity of detection. Histological localization of Epacadostat, Trp and Kyn was shown on CT26 tumors using MSI analysis (FIG. 10A). Molecular images showed higher Kyn and lower Trp levels on control CT26 tissue compared to treated tissue. Afterwards, for the QMSI, the same derivatization strategy was used to perform both normalized Trp and Kyn calibration curves (FIG. 11A). Absolute quantification using QMSI and LC-MS/MS analysis of both Kyn and Trp on control and treated CT26 tissue sections followed by a Kyn/Trp ratio calculation were plotted on FIG. 9B that showed a good correlation between both technologies. A decrease of Kyn/Trp ratio was noticed after EPA treatment and a 6 folds decrease was found whatever used technology. Plasma and blood samples were also analyzed and results showed a Kyn/Trp ratio decrease of 3 folds when treated (FIG. 11B).

2.4. From Target Exposure to Response Efficacy Analysis: EPA Regional/Pharmacological Effect on CT26 Tumor Mouse Model

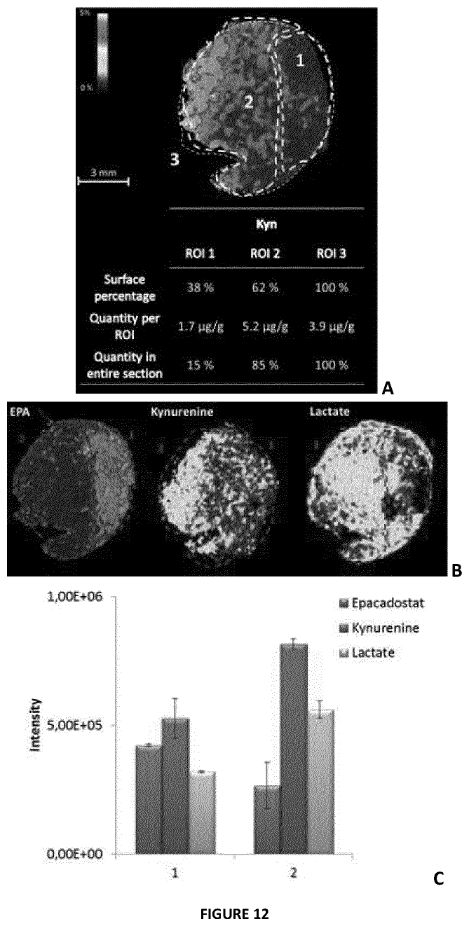

Pharmacology efficacy of EPA drug was followed through the absolute quantification of Kyn metabolite as direct enzymatic product of IDO1 enzyme. Absolute Kyn quantity in the three ROIs showed the presence of 15% of Kyn in 38% of the entire tumor (ROI 1) and 85% in ROI 2 (FIG. 12A). Finally, histological distribution of Lactate metabolite as EPA and Kyn were showed in FIG. 12B, followed by a plot for which relative intensities of all molecules were extracted from every x and y position of high and low EPA regions (1 and 2, respectively). Negative correlation was so found between EPA, Kyn and Lactate metabolites, since high EPA level on ROI 1 corresponded to low expression level of both Kyn and Lactate (FIG. 12C).

### Analysis of the Results

The results of the study showed a quantity between 6.6+/−1 μg/g and 7.3+/−2 μg/g in the plasma and the blood, respectively (FIG. 7C). Then, using LC-MS/MS and QMSI of treated CT26 tumors, the available on tissue quantity of EPA was 3 or 4 times higher than the plasma (between 35 and 48 μg/g) with a variation of less than 20% between both techniques. Related to the EPA inhibitory effect, kynurenine level decrease was noticed in plasma in a dose-dependent fashion. In this experiment, >50% suppression was seen for approximately 24 hours with the 100 mg/Kg dose over the course of the day.

Our last study, showed a decrease of Kyn level on P815 mouse models from 25-34 μg/g (P815 high IDO1) to <60 ng/g (P815 low IDO1).

In this study, CT26 murine colon carcinoma cells were well known to express IDO1, and therefore was used for determining the effects of IDO1 inhibition on tumor growth. Indeed, CT26 model was already well used for the pharmacological evaluation of IDO1 inhibitors. Historically, the abundance and distribution of drugs have been assessed by well-established techniques such as quantitative whole-body autoradiography (QWBA) or tissue homogenization with LC-MS/MS analysis. However, QWBA does not distinguish active drug from its metabolites and LC-MS/MS, while highly sensitive, does not report spatial distribution. Mass spectrometry imaging (MSI) can discriminate drug and its metabolites and endogenous compounds, while simultaneously reporting their distribution. MSI data are influencing drug development and currently used in investigational studies in areas such as DMPK, PD and toxicity.

The present study showed the high impact of using QMSI technology for a target exposure research purpose. When comparing control and treated CT26 tumors, specific regions were segmented regarding the EPA quantity contained inside. Tumor exposure to EPA was so confirmed than two distinguishable regions were extracted (1 for high and 2 for low EPA). Almost, 61% of EPA drug corresponding to 68 μg/g was localized in 38% of the entire tumor. Semi-quantitative analysis of IDO1 enzyme showed a high expression of IDO1 in ROI 1 compared to ROI 2, what came supporting the EPA exposure to its IDO1 target.

Since Kyn and Trp basal levels were pretty low, a derivatization step was initiated to allow the on-tissue localization of endogenous metabolites. As MALDI MSI is used for the multiplex detection of diverse analytes over a wide mass range, analyte coverage is highly limited to the more abundant compounds. On-tissue or in-tissue analyte chemical derivatization addresses these issues by selectively tagging functional groups specific to a class of analytes, while simultaneously changing their molecular masses and improving their ionization efficiency and so their sensitivity. Therefore, Trp and Kyn sensitivities increased allowing their on-tissue distribution and quantification studies. Several studies showed that sera from cancer patients have higher Kyn/Trp ratios than sera from normal volunteers, consistent with increased IDO1 activity. Using both QMSI and LC-MS/MS, our data showed a Kyn/Trp ratio decrease of 6 and 3 times when comparing control to treated CT26 tissues, plasma and blood, respectively (FIG. 11B).

Similarly, Kyn/Trp ratio was recently validated as a prognostic tool in many cancers: cervical, glioblastoma, and lung, where LC-MS/MS was used for the metabolites quantification. Then, compared to a region from where EPA is absent (control tumor), Kyn expression was extracted showing a regional correlation between the EPA presence and its pharmacological effect on Kyn.

Finally, IDO1 enzyme immunostaining was realized allowing seeing the EPA target histological localization. More than the substrate and product alterations linked to IDO1 inhibition, cancer cells exhibited metabolic alterations that distinguished them from healthy tissues and made their metabolic processes susceptible to pharmacological targeting. Cellular metabolites have vastly diverse physicochemical properties, thereby necessitating the combination of various analytical methods for their detection and quantification. The presence of a specific metabolite may inform of the metabolic state of a tumor; however, it is not always straightforward to infer the activity of specific metabolic pathways from the measurement of metabolite abundances alone.

The results of the present study showed a histological anti-localization between Lactate and EPA molecules. As Lactate is the final product of the glycolysis pathway, its decrease corresponded to a marked glycolysis decrease on the same histological region where EPA was highly concentrated. Assessing the regional level of metabolites such as Lactate and glucose was performed using quantitative bioluminescence imaging for ischemia in brain flash-frozen biopsies.

Across a wide variety of tumors and types of cancer, Lactate was demonstrated to be a prognostic indicator, since its elevated level correlated with poorer patient prognosis, poor disease-free or metastasis-free survival and poor overall survival in human cervical cancers, head and neck cancer, high-grade gliomas and non-small-cell lung cancer. This feature makes Lactate metabolism of interest for further investigations, not only as a biological marker, but also as a potential therapeutic end point or target.

Subsequently, by inhibiting IDO1 and decreasing Kyn in tumor cells, EPA restored the proliferation, activation and regulation of various cells. Indeed, this would support the EPA pharmacological effect. Moreover, TCA cycle and energetic pathway metabolism was also highlighted using MALDI MSI, showing different histological localizations and relative intensities (data not shown). The presence of a specific group of metabolites may inform of the metabolic state of a tumor. Extracellular tumor microenvironment characterization was realized showing glycolysis signature such as Lactate, glucose, malate, citrate, glutamine and proline. A prevailing picture was that, in tumors, a higher fraction of glucose carbons was diverted away from mitochondria and converted to Lactate. Also, glutamine metabolism and function in relation to proline synthesis is shown. Finally, degradation of purines nucleotides was also shown.

### 3. CONCLUSION

To conclude, these results reported a quantitative ultrahigh mass resolution study that allowed highlighting an in-situ PK/PD study of the EPA. Indeed, different studies were realized:
Bio-distribution and bioavailability study was performed through on-tissue drug quantification.
Targeted tissue exposure study was done after tissue segmentation based on biomarker and/or drug histological localization.
Pharmacodynamics study was also performed showing a regional drug exposure to response effect, which made an efficacy analysis possible.

Indeed, as exposure at the site of action and to its specific target were identified as the most important factors for success in drug discovery and the design of chemical probes, these results showed and confirmed the high contribution of QMSI, and more generally MSI, to study the relationship between target occupancy and drug efficacy.

1-15. (canceled)

16. A method for ex-vivo or in-vitro evaluation of an effect of at least one molecule of interest on at least one molecular marker in a dosed biological sample comprising:
selecting a dosed biological sample, which has been previously exposed to said at least one molecule of interest;
detecting the presence of the at least one molecule of interest in the dosed biological sample with a molecular imaging method and obtaining a molecular map of the dosed biological sample for the at least one molecule of interest;
spatially segmenting the molecular map of the dosed biological sample as a function of spectral information to obtain a segmentation map of the at least one molecule of interest in the dosed biological sample;
selecting a first region of interest (ROI) from the segmentation map, said first ROI having a first intensity for the at least one molecule of interest;
measuring the intensity or quantity of at least one molecular marker in said first ROI;
comparing the intensity or quantity of at least one molecular marker in the first ROI with the intensity or quantity of at least one molecular marker in a second ROI, said second ROI being selected from the dosed biological sample or from another biological sample.

17. The method of claim 16, wherein the biological sample is selected from the group consisting of a tissue sample, organoids, and a biological fluid sample.

18. The method of claim 16, wherein the dosed biological sample is a tissue section which has been obtained by previously sampling an animal which has been previously administered by the molecule of interest, or wherein the dosed biological sample has been contacted in vitro with the molecule of interest.

19. The method of claim 16, wherein the molecular imaging method is selected from Mill imaging, PET imaging, CT imaging, IF, ISH, IHC, mass spectrometry or cytometry imaging.

20. The method of claim 16, wherein the molecule of interest is a candidate molecule and the dosed biological sample has been previously obtained by sampling in an animal that has been previously administered with the candidate molecule.

21. The method of claim 16, wherein the second ROI is selected from the segmentation map of the dosed biological sample, said second ROI being physically different from the first ROI and having a second intensity for the molecule of interest.

22. The method of claim 16, wherein the second ROI is selected from a second biological sample which has not been previously exposed to the molecule of interest, said second biological sample being from same biological origin as the dosed biological sample, and wherein optionally the comparison between the first and second ROI allows to identify at least one biological marker specific to the molecule of interest.

23. The method of claim 16, wherein the second ROI is selected from a second biological sample, which has been previously exposed to the molecule of interest at a dose concentration different from the dose concentration for the dosed biological sample, said second biological sample being from same biological origin as the dosed biological sample.

24. The method of claim 16, wherein the second ROI is selected from a second biological sample which has been previously exposed to a second molecule of interest, said second biological sample being from same biological origin as the dosed biological sample.

25. The method of claim 24, wherein the molecules of interest are two distinct candidate molecules, the comparison between the first ROI and the second ROI allows to discriminate between said candidate molecules.

26. The method of claim 24, for evaluating and comparing the effect of at least two different molecules of interest on at least one molecular marker.

27. The method of claim 16, wherein the measure of the quantity or intensity of the biological marker in the first ROI and/or the second ROI is performed with a molecular imaging method and/or wherein the measure of the quantity or intensity of the biological marker in the first ROI and/or the second ROI is performed by bioanalysis.

28. The method of claim 16, wherein the ROI is extracted from the biological sample by laser capture microdissection, and wherein the ROI is optionally cultivated in a culture medium before performing the bioanalysis.

29. The method of claim 28, wherein an effect of the molecule of interest is evaluated by comparing gene or transcripts expression, lipidomics, peptidomics, proteomics and/or metabolics changes between the first and the second ROI.

30. The method of claim 16, wherein the molecule of interest is a therapeutic antibody, and wherein the presence of said therapeutic antibody is detected by contacting the biological sample with a marked antibody anti-therapeutic antibody.

31. The method of claim 19, wherein the molecular imaging method and is selected from MALDI, DESI, LESA, LA-ICP-MS or SIMS.

32. The method of claim 27, wherein the molecular imaging method is selected from MSI, ISH, IF, IHC, MRI, imaging mass cytometry, CT or PET imaging.

33. The method of claim 27, wherein the bioanalysis is performed by mass spectrometry, electrophoresis, ligand binding assay, nuclear magnetic resonance, microdialysis and chromatography, magnetic bead multiplex immunoassay or ELISA.

34. The method of claim 17, wherein the biological sample is a urine sample, plasma sample, cerebrospinal fluid, or a cell suspension.
